(19) United States
(12) Patent Application Publication
(10) Pub. No.: US 2024/0158614 A1
(43) Pub. Date: May 16, 2024

(54) STORAGE STABLE SELF-ADHESIVE COMPOSITE CEMENTS WITH GOOD TRANSPARENCY AND GOOD RADIOPACITY

(71) Applicant: Ivoclar Vivadent AG, Schaan (LI)

(72) Inventors: Alexandros Gianasmidis, Balgach (CH); Yohann Catel, Sevelen (CH); Norbert Moszner, Triesen (LI); Silke Siegriest, Mandelbachtal (DE); Budimann Ali, Saarbrücken (DE); Carsten Becker-Willinger, Saarbrücken (DE); Marek Twardoch, Sarreguemines (FR)

(21) Appl. No.: 18/490,396

(22) Filed: Oct. 19, 2023

(30) Foreign Application Priority Data
Oct. 20, 2022 (EP) 22202860.7

(51) Int. Cl. C08K 9/04; A61C 5/00; C08F 222/10; C08F 222/38; C08K 5/09; C09C 1/30; C09C 3/00; C09C 3/06; C09C 3/08

(52) U.S. Cl. CPC C08K 9/04; A61C 5/00; C08F 222/102; C08F 222/385; C08K 5/09; C09C 1/3054; C09C 1/3063; C09C 3/006; C09C 3/063; C09C 3/08; A61C 2201/002; C08F 2800/20

(57) ABSTRACT

A radically polymerizable composition comprising at least one polyfunctional radically polymerizable monomer having no acid group, at least one acid group-containing radically polymerizable monomer and/or at least one acidic oligomer, at least one fluoroaluminosilicate glass filler and/or radiopaque glass filler, and at least one initiator for the radical polymerization, wherein the fluoroaluminosilicate glass filler and/or radiopaque glass filler is coated with a silica (hetero)polycondensate. The composition is characterized by high stability and is particularly suitable as a dental material.

### CROSS-REFERENCE TO RELATED APPLICATIONS

This application claims priority to European Patent Application No. 22202860.7 filed on Oct. 20, 2022, the disclosure of which is incorporated herein by reference in its entirety.

### TECHNICAL FIELD

The present invention relates to radically polymerizable self-adhesive composites with good transparency and radiopacity, which are characterized by high storage stability and are particularly suitable as dental materials, especially as dental cements, filling composites or veneering materials, and for the fabrication of inlays, onlays, or crowns.

### BACKGROUND

Composites are mainly used in the dental field for the fabrication of direct and indirect fillings, i.e. as direct and indirect filling composites, and as cements. The polymerizable organic matrix of the composites usually consists of a mixture of monomers, initiator components and stabilizers. Mixtures of dimethacrylates are usually used as monomers, which may also contain monofunctional and functionalized monomers. Commonly used dimethacrylates are 2,2-bis[4-(2-hydroxy-3-methacryloyloxypropyl)-phenyl]propane (Bis-GMA), 1,6-bis[2-methacryloyloxyethoxycarbonylamino]-2,2,4-trimethylhexane (UDMA), which have high viscosity and give polymers with good mechanical properties and low polymerization shrinkage. Triethylene glycol dimethacrylate (TEGDMA), 1,10-decanediol dimethacrylate (D₃MA) or bis(3-methacryloyloxymethyptricyclo[5.2.1.0^{2,6}]decane (DCP) are mainly used as reactive diluents. Monofunctional methacrylates, such as p-cumylphenoxyethylene glycol methacrylate (CMP-1E), are also suitable for reducing viscosity and in addition cause a reduction in network density and increased double bond conversion.

To produce self-adhesive composites, strongly acidic adhesive monomers are used, such as 10-methacryloyloxydecyl dihydrogen phosphate (MDP), which etches the tooth structure and causes adhesion to enamel/dentin by ionic interaction. Adhesive monomers impart self-adhesive properties to composites and thus enable the composites to be used without pretreatment of the tooth structure with an enamel/dentin adhesive, which makes their use particularly attractive.

In addition to the organic matrix, composites contain one or more fillers, which are usually surface-modified with a polymerizable coupling agent, such as 3-methacryloyl-oxypropyltrimethoxysilane. Fillers improve the mechanical properties (strength, modulus of elasticity, abrasion resistance) and the processing properties (paste consistency, sculptability) of the materials and impart radiopacity.

It is problematic that acidic adhesive monomers often interact adversely with fillers. For example, the acidic adhesion monomers are bound to the surface of the fillers by the formation of insoluble salts, or they form poorly soluble salts during storage with ions released from the fillers. This leads to a significant reduction of the adhesion monomer concentration in the resin matrix, which is associated with a reduction or even a loss of the adhesion properties. Composites with acidic adhesive monomers therefore have only limited storage stability.

Methacrylate-based dental materials are cured by radical polymerization, using radical photoinitiators, thermal initiators or redox initiator systems, depending on the field of application. Dual-curing systems contain a combination of photoinitiators and redox initiators.

Composite cements usually contain redox initiator systems because they ensure sufficient curing even when light curing is not possible due to insufficient transmittance. Redox initiator systems based on a mixture of dibenzoyl peroxide (DBPO) with tertiary aromatic amines, such as N,N-diethanol-p-toluidine (DEPT), N,N-dimethyl-sym.-xylidine (DMSX) or N,N-diethyl-3,5-di-tert-butylaniline (DABA), are usually used. Since radical formation in DBPO/amine-based redox initiator systems is greatly impaired by strong acids and thus also by strongly acidic adhesive monomers, cumene hydroperoxide-containing redox initiator systems in combination with thioureas, such as acetylthiourea, are preferred.

In order to ensure sufficient storage stability of the redox initiators, redox initiator system-based materials are usually used as so-called 2-component systems (2C), whereby the oxidizing agent (peroxide or hydroperoxide) and the reducing agent (amines, sulfinic acids, barbiturates, thioureas, etc.) are incorporated into spatially separated components. These are mixed together only shortly before use. For mixing, double-push syringes are increasingly used, which have separate cylindrical chambers to hold the components. The components are pushed out of the chambers simultaneously by two interconnected pistons and mixed together in a nozzle. To obtain mixtures that are as homogeneous as possible, it is advantageous to mix the components together in approximately equal volume proportions.

Conventional luting cements, such as ZnO eugenol cements, zinc phosphate cements, glass ionomer cements (GIC) and resin modified glass ionomer cements (RMGI), are not suitable for use with double-push syringes because they contain a powder component, which makes mixing of the components considerably more difficult. In addition, glass ionomer cements have only low transparency and relatively poor mechanical properties.

Conventional glass ionomer cements (GIC) contain an aqueous solution of a high molecular weight polyacrylic acid (PAA, number average molar mass greater than 30,000 g/mol) or a copolymer of comparable molar mass of acrylic acid and itaconic acid as liquid component and a calcium fluoro-alumino glass as powder component. After mixing the components, they cure by purely ionic ionomer formation.

Resin modified glass ionomer cements (RMGI) contain additionally hydrophilic monomers, such as 2-hydroxyethylmethacrylate (HEMA). They cure both by an acid-base reaction and by radical polymerization. Compared to conventional GIC they are characterized by improved flexural strength.

US 2015/0013568 A1 discloses the coating of dental barium and strontium glass fillers with an acid-resistant oxide, for example with SiO₂ powder, to improve the acid resistance of the fillers. This process is said to yield dense glass particles.

WO 2013/156185 A1 discloses water-soluble oligomers obtained by hydrolysis and condensation of aminosilanes and subsequent reaction of the reaction products with an acrylic anhydride. Direct reaction of aminosilanes with acrylic anhydride is said to produce undesirable transesterification products, which require complex purification of the products. The formation of transesterification products is said to be avoided by prior hydrolysis and condensation of the aminosilanes to oligomers. The oligomers are said to be suitable, among other things, as adhesion promoters or for coating fillers, for example in dental impression materials and dental acrylics.

### SUMMARY

It is an object of the present invention to provide self-adhesive dental composites with good transparency, good mechanical properties and high radiopacity, which, as 2-component systems, can be mixed and applied well with double-push syringes. The composites are to be particularly suitable as dental luting cements and exhibit high storage stability.

### BRIEF DESCRIPTION OF THE DRAWINGS

Exemplary embodiments of the invention are shown in the drawings and are described in more detail below, in which:

FIG. 1 shows the particle size distribution of fillers coated according to the invention in comparison with the uncoated starting materials;

FIG. 2 shows the particle size distribution of fillers coated according to the invention in comparison with the uncoated starting materials;

FIG. 3 shows the particle size distribution of fillers coated according to the invention in comparison with the uncoated starting materials;

FIG. 4 shows the decrease in the concentration of the acidic monomer MDP as a function of the storage time in a composite paste with the coated glass filler; and

FIG. 5 shows the decrease in the concentration of the acidic monomer MDP as a function of storage time in a composite paste with the coated glass filler; and

FIG. 6 shows the dentin shear bond strength of a two-component composite cement with the coated filler.

### DETAILED DESCRIPTION

The object of the invention is achieved by filler-containing, free-radically polymerizable compositions comprising at least one free-radically polymerizable monomer, at least one acidic, free-radically polymerizable monomer, at least one fluoroaluminosilicate glass filler (FAS filler) and/or radiopaque glass filler and at least one initiator for the free-radical polymerization. The compositions are characterized in that the filler is coated with a silica (hetero)polycondensate. The term silica (hetero)polycondensate stands for silica polycondensate or silica hetero polycondensate. Surprisingly, it was found that coating the filler with a silica (hetero)polycondensate results in a significant increase in the storage stability of the compositions without deteriorating the other properties. Compositions containing fillers are referred to as composites.

The filler is preferably coated by a sol-gel process. For this purpose, a sol is prepared by hydrolytic condensation of one or more condensable compounds of silicon and optionally other elements from the group boron, aluminum, titanium or phosphorus and/or precondensates derived from these compounds, brought into contact with the filler and the filler is subsequently dried.

Preferred hydrolytically condensable compounds of silicon are silanes of the general formula I:

SiXₐR₄₋ₐ Formula I

in which the variables have the following meanings:
R=C₁-C₂₀-alkyl or C₂-C₁₀-alkenyl, where alkyl and alkenyl radicals may comprise one or more O or S atoms, ester, amide, amino or urethane groups, C₆-C₁₄-aryl or C₇ -C₁₅-arylalkyl;
X=halogen, hydroxy, C₁-C₁-alkoxy, C₁-C₆-acyloxy;
a=2, 3 or 4, preferably 3 or 4.

If there are multiple R or X residues, they may be different or preferably the same.

Arylalkyl groups are hydrocarbon groups containing at least one aromatic and at least one non-aromatic residue, such as a —CH₂—CH₂-Ph or a -Ph-CH₃ group with Ph=phenyl.

Particularly preferred are silanes of the formula I in which at least one and preferably all variables have one of the following meanings:
R=C₁-C₁₀-alkyl or C₂-C₈-alkenyl, where alkyl and alkenyl radicals may comprise one or more O atoms or ester groups, phenyl or C₇-C₁₅-arylalkyl;
X=hydroxy, C₁-C₅-alkoxy-, C₁-C₃-acyloxy;
a=3 or 4.

The silanes of formula I can be hydrolytically condensed via the radicals X to form an inorganic network with Si—O—Si units. If the radicals R contain radically polymerizable groups, an organic network can also be formed via them.

More preferred are silanes of the formula I where the variables have the following meanings:
R methyl, ethyl, n-propyl, i-propyl, n-butyl, s-butyl, n-pentyl, vinyl, a (meth)acryl group (H₂C═C(CH₃)—COO—), allyl, phenyl or naphthyl, particularly preferred are ethyl, n-propyl, i-propyl and most preferred are methyl or 3-(meth)-acryloyloxypropyl;
X methoxy, ethoxy, n-propoxy, i-propoxy, n-butoxy, F, Cl, Br, especially preferably Cl and most preferably ethoxy;
a 3 or 4.

According to the invention, silanes of the formula SiX₄ (a=4) are particularly preferred, with Si(OCH₃)₄ and Si(OC₂H₅)₄ being even more preferred. Silanes of the formula SiX₄ give a particularly high network density. They are preferably used in an amount of 40 to 100% by weight, more preferably 50 to 100% by weight and most preferably 60 to 95% by weight, based on the total amount of silanes of formula I.

Further preferred are silanes of formula I containing at least one free-radically polymerizable group, 3-methacryloyloxypropyltrimethoxysilane being particularly preferred. The radically polymerizable group enables covalent bonding between the coated fillers and the radically polymerizable matrix during curing of the materials according to the invention. Silanes containing radically polymerizable groups are preferably used in an amount of 0 to 10% by weight, more preferably 0 to 8% by weight and most preferably 1 to 5% by weight, based on the total amount of silanes of the formula I.

In addition to the silane(s) of formula I, one or more further hydrolytically condensable compounds of the elements Al, Ti, B or P can be used to coat the fillers. Preferred compounds are those of Al, Ti and B. Preferred aluminum compounds are aluminum sec-butylate and aluminum isopropylate. Preferred titanium compounds are Ti(OC₂H₅)₄, Ti(OC₃H₇)₄, Ti(O-iC₃H₇)₄ and Ti(OC₄H₃)₄. Preferred boron compounds are B(OCH₃)₃ and B(OC₂H₅)₃ . By using additional hydrolytically condensable compounds, the properties of the coating, such as hardness and abrasion resistance, can be specifically controlled. One or more additional compounds may be used. These are preferably used in a total amount of 0 to 20% by weight, more preferably 0 to 15% by weight and most preferably 0 to 10% by weight, based on the total amount of silanes of formula I. Metal alkoxides, for example of Al or Ti, can also be used in complexed form, for example in the form of the reaction products of Al or Ti alkoxides with (meth)acrylic acid or 2-acetoacetoxyethyl methacrylate. The proportion of silanes of the formula I is preferably 80 to 100% by weight, more preferably 85 to 100% by weight and most preferably 90 to 100% by weight, based on the total amount of hydrolytically condensable compounds.

The silane or silanes of formula I and optionally other hydrolytically condensable compounds are hydrolyzed and condensed in the presence of water, preferably at least the amount of water stoichiometrically required for hydrolysis, and optionally a condensation catalyst and/or a solvent. Polycondensates or heteropolycondensates are formed in this process. Instead of the hydrolytically condensable compounds, precondensates derived therefrom can also be used. Precondensates are reaction products of the alkoxysilanes and/or halosilanes and optionally of the metal alkoxides with water with partial or complete elimination of the alcohols or hydrogen halides. In the case of the alkoxysilanes, silanols are formed as precondensates.

Preferably, the hydrolytic condensation is carried out by dispersing the filler to be coated in the silane(s) of formula I and, if desired, further hydrolytically condensable compounds or, preferably, a solution of the silane(s) and, if desired, further hydrolytically condensable compounds in a suitable solvent and then adding water. The mixture is preferably stirred in this process.

If only silanes are used, the hydrolysis is preferably carried out at room temperature or under slight cooling. Preferably, a hydrolysis or condensation catalyst is added. Polycondensates are obtained in this process. The resulting mixture (dispersion) is preferably stirred for several hours to several days at a temperature of -20 to 150° C., preferably 20 to 110° C. The filler is then separated, e.g. by centrifugation, then optionally washed with a solvent one or more times and then dried. Drying can be carried out at room temperature or at elevated temperature, preferably at 50 to 60° C. Preferably, the filler is dried in a vacuum drying cabinet. Drying may extend over a period of several hours to several days. Preferably, drying is carried out until the weight is constant. After drying, the fillers may be subjected to further heat treatment. This is preferably carried out at a temperature of from 20 to 150° C., more preferably from 30 to 100° C. and most preferably from 40 to 80° C. Temperature and treatment time are selected so that any organic groups present in the coating are not destroyed and no glass layer is formed.

If, in addition to the silanes of formula I, other hydrolytically condensable compounds are used to coat the filler, the water is preferably added in stages at -20 to 100° C., preferably at 0 to 30° C. In this case, heteropolycondensates are formed. The method of water addition depends primarily on the reactivity of the hydrolytically condensable compounds used. The desired amount of water can be added, for example, in portions or in one portion.

In the preparation of both polycondensates and heteropolycondensates, the water is preferably added in an amount of 0.5 to 10 moles of water per mole of hydrolyzable groups, particularly preferably in an amount of 1 to 10 moles of water per mole of hydrolyzable groups and most preferably in an amount of 1.5 to 5 moles of water per mole of hydrolyzable groups of the silanes of formula I and optional other hydrolytically condensable compounds. The water or portions thereof may be introduced in pure form or as a constituent of other components, e.g. of the catalyst solution.

Preferred solvents for performing the hydrolytic condensation are lower aliphatic alcohols, such as ethanol or i-propanol, lower dialkyl ketones, such as acetone or methyl isobutyl ketone, cyclic ethers, such as tetrahydrofuran or dioxane, aliphatic ethers, such as isopropoxyethanol or isopropoxypropanol, amides, such as dimethylformamide, and mixtures thereof.

Preferred hydrolysis or condensation catalysts are organic or inorganic acids, such as hydrochloric acid, sulfuric acid, p-toluenesulfonic acid, formic acid or acetic acid. Ammonium fluoride and organic bases, in particular amines, such as n-propylamine, diethylamine or triethylamine, and aminosilanes, such as 3-aminopropyltrimethoxy-silane, are further preferred.

The condensation time depends on the respective starting components, their proportions, the catalyst used, if any, the reaction temperature, etc. Preferably, the reaction time is in the range of 1 to 96 h, more preferably 2 to 72 h and most preferably 3 to 48 h.

When drying the fillers and evaporating the solvent, the hydrolysis and condensation reaction leads to the formation of a gel film via the formation of sol particles, which further cross-links in the course of drying and forms a dense inorganic network on the filler surface. The properties of the coating, such as strength, flexibility, adhesion, hardness, refractive index and impermeability, can be specifically adjusted by the selection of the silanes and the other hydrolytically condensable components and adapted to the particular application. For example, the functionality of the silanes of formula I, which can vary between 4, e.g. in tetraethoxysilane (TEOS), and 2, e.g. in dialkoxysilane, can be used to adjust the degree of crosslink-linking and thus the strength, flexibility and impermeability of the coating. By selecting the organic groups of the silanes, it is possible to specifically influence, for example, the flexibility, polarity, wetting or thickening effect of the coating. Silanes with polymerizable groups, such as in 3-methacryloyloxypropyltrimethoxysilane, allow additional crosslinking of the coating by radical polymerization and covalent incorporation of the fillers into the polymer matrix. Co-condensation with metal alkoxides, such as Ti alkoxide, allows control of the kinetics of the hydrolytic condensation and, for example, an increase in density and hardness as well as adjustment of the refractive index of the coating.

The filler is preferably dispersed in the hydrolysis mixture in an amount of from 1 to 25% by weight, more preferably from 3 to 20% by weight and most preferably from 4 to 15% by weight, based on the total mass of the mixture. According to the invention, particulate fillers are preferred.

Fluoroaluminosilicate glass fillers (FAS fillers) and radiopaque glass fillers are preferred as fillers to be coated.

Particularly preferred radiopaque glass fillers have the following composition (wt %r): SiO₂: 20-80; B₂O₃: 5-15; BaO or SrO: 0-30; Al₂O₃: 5-20; CaO and/or MgO: 0-20; Na₂O, K₂O, Cs₂O: 0-10 each; WO₃: 0-20; La₂O₃: 0-10; ZrO₂: 0-15; P₂O₅: 0-10; Ta₂O₅, Nb₂O₅ or Yb₂O₃: 0-5 and CaF₂ or SrF₂: 0-10. Very particularly preferred are radiopaque glass fillers having the composition (wt. %); SiO₂: 50-75; B₂O₃: 5-15; BaO or SrO: 2-30; Al₂O₃: 5-15; CaO and/or MgO: 0-10 and Na₂O: 0-10.

Particularly preferred FAS fillers have the following composition (wt %): SiO2: 20-35; Al₂O₃: 15-35; BaO or SrOl 0-25; CaO: 0-20; ZnO: 0-15; P₂O₅: 5-20; Fluoride: 3-18. Very particularly preferred FAS fillers have the composition (wt %): SiO₂: 20-30; Al₂O₃: 20-30; BaO or SrO: 15-15; CaO: 5-18; P₂O₅: 5-15; Na₂O: 2-10; and CaF₂: 5-20.

All percentages are based on the total mass of the glass, with the components except fluorine being calculated as oxides, as is common for glasses and glass ceramics, and do not include the coating.

According to the invention, particulate FAS fillers and radiopaque glass fillers with a mean particle size of 0.2 to 20 μm are preferred and with a mean particle size of 0.4 to 5 μm are particularly preferred. The size specifications do not include the coating. The layer thickness of the coating is preferably in a range of 5 to 500 nm.

Unless otherwise stated, all particle sizes herein are volume averaged particle sizes (D50 values), i.e., 50% of the total volume of all particles is contained in particles having a diameter smaller than the indicated value.

Particle size determination in the range from 0.1 μm to 1000 μm is preferably carried out by means of static light scattering (SLS), for example with an LA-960 static laser scattering particle size analyzer (Horiba, Japan) or with a Microtrac S100 particle size analyzer (Microtrac, USA). Here, a laser diode with a wavelength of 655 nm and an LED with a wavelength of 405 nm are used as light sources. The use of two light sources with different wavelengths enables the measurement of the entire particle size distribution of a sample in only one measurement run, whereby the measurement is carried out as a wet measurement. For this purpose, an aqueous dispersion of the filler is prepared and its scattered light is measured in a flow cell. The scattered light analysis for calculating particle size and particle size distribution is carried out according to the Mie theory according to DIN/ISO 13320. The measurement of the particle size in a range from 1 nm to 0.1 μm is preferably carried out by dynamic light scattering (SLS) of aqueous particle dispersions, preferably with a He—Ne laser with a wavelength of 633 nm, at a scattering angle of 90° and at 25° C., e.g. with a Malvern Mastersizer 2000 with dispersion unit Hydro 2000S (Malvern Instruments, Malvern UK).

In the case of aggregated and agglomerated particles, the primary particle size can be determined from TEM images. Transmission electron microscopy (TEM) is preferably performed using a Philips CM30 TEM at an accelerating voltage of 300 kV. For sample preparation, drops of the particle dispersion are applied to a 50 Å thick copper grid (mesh size 300 mesh) coated with carbon, followed by evaporation of the solvent. The particles are counted and the arithmetic mean is calculated.

The radiopaque glass fillers or FAS fillers can be pretreated with acid before coating. The acid treatment of the particulate FAS or glass fillers is preferably carried out by washing the filler with acid, particularly preferably by the following process:
(i) The particulate FAS or glass filler is dispersed in an aqueous solution of an organic or preferably inorganic acid. The acid is preferably used in a concentration of 0.1 to 5 mol/l, more preferably 0.5 to 3 mol/l.
(ii) Subsequently, the dispersion from step (i) is stirred, preferably for 0.5 to 24 h, more preferably for 1 to 5 h.
(iii) After stirring in acid solution, the filler is separated and washed with deionized water, For this purpose, it is preferably dispersed in water and stirred for 1 to 60 minutes, more preferably 2 to 20 minutes.
(iv) After washing, the filler is separated and dried, preferably in a vacuum drying cabinet in a fine vacuum at 20 to 80° C., more preferably at 40 to 60° C. The filler is preferably dried to constant weight.
(v) After drying, the filler is preferably subjected to optional annealing. For this purpose, it is preferably heated for 2 to 12 h, particularly preferably 4 to 6 h, to a temperature which is well below the glass transition temperature of the filler, preferably 200 to 500° C. and particularly preferably 300 to 400° C.

The acid used to treat the fillers is preferably completely removed from the fillers after the acid treatment. The fillers are not coated with an acid.

The acid treatment can be repeated once or several times. Steps (i) and (iii) are preferably carried out at a temperature in the range from 5 to 50° C., particularly preferably at room temperature (23° C.). The temperature is measured in the solution or dispersion in each case.

Preferred are acids that form soluble salts with Ca, Al, Sr and Ba ions. Particularly preferred are formic acid, acetic acid and especially hydrochloric acid, and nitric acid. Alternatively, acids that form sparingly soluble salts with Ca, Al, Sr or Ba ions, such as phosphoric acid, can be used, but these are less preferred. Poorly soluble salts are defined as salts with a solubility of less than 0.1 g/l (in water at room temperature). Acidic organic monomers and acidic organic polymers as well as peracids and hydrofluoric acid are not suitable as acids according to the invention.

The washing step (iii) is preferably repeated 1 to 5 times, more preferably 3 times, so that the acid is completely removed from the filler. For this purpose, the filler is separated from the water following step (iii) and again dispersed and stirred in deionized water. The washing is repeated until the pH of the water in the last washing step is ≥5.

After washing, the filler can be subjected to further acid treatment and washing. The sequence of acid treatment and washing can be repeated once or several times.

The coating of the fillers according to the invention prevents significant amounts of metal ions, in particular Al, Ca, Ba or Sr ions, from diffusing from dental fillers, such as radiopaque glass fillers and FAS fillers, into the resin mixture and reacting there with acidic monomers. It also prevents acidic monomers from binding to the filler surface by forming insoluble salts. This avoids a decrease in the concentration of acid monomers in the resin matrix and a consequent deterioration in adhesion, and improves the storage stability of the materials.

In addition to the coated FAS and radiopaque glass fillers, the compositions according to the invention may contain other fillers.

Preferred further fillers are metal oxides, particularly preferably mixed oxides, which contain 60 to 80% by weight of SiO₂ and at least one of the metal oxides ZrO₂, Yb₂O₃, ZnO, Ta₂O₅, Nb₂O₅ and/or La₂O₃, preferably ZrO₂, Yb₂O₃ and/or ZnO, so that the total amount adds up to 100%. Mixed oxides such as SiO₂—ZrO₂ are accessible, for example, by hydrolytic co-condensation of metal alkoxides. The metal oxides preferably have a mean particle size of 0.05 to 10 μm, particularly preferably 0.1 to 5 μm. The metal oxide or oxides are preferably also coated in the manner described above.

Other preferred additional fillers are fumed silica or precipitated silica with a primary particle size of 0.01 to 0.15 μm, and quartz or glass ceramic powder with a particle size of 0.1 to 15 μm, preferably from 0.2 to 5 μm, and ytterbium trifluoride. The ytterbium trifluoride preferably has a particle size of 80 to 900 nm and more preferably 100 to 300 nm. These fillers are preferably used in an amount of 0.1 to 25% by weight, more preferably 0.2 to 20% by weight and most preferably in an amount of 0.3 to 15% by weight, in each case based on the total mass of the composition.

In addition, so-called composite fillers are preferred as further fillers. These are also referred to as isofillers. These are splinter-shaped polymers which in turn contain a filler, preferably fumed SiO₂, glass filler and/or ytterbium trifluoride. Preferred are polymers based on dimethacrylates. For the production of isofillers, the filler(s) is/are incorporated, for example, into a dimethacrylate resin matrix, and the resulting composite paste is subsequently thermally polymerized and then ground.

A composite filler preferred according to the invention can be prepared, for example, by thermally curing a mixture of Bis-GMA (8.80 wt. %), UDMA (6.60 wt. %), 1,10-decanediol dimethacrylate (5.93 wt. %), dibenzoyl peroxide+2,6-di-tert.butyl-4-methylphenol (together 0.67 wt. %), glass filler (average particle size 0.4 μm; 53.0 wt. %) and YbF₃ (25.0 wt. %) and then grinding the cured material to the desired particle size. All percentages refer to the total mass of the composite filler.

To improve the bond between filler and matrix, the fillers are preferably surface modified with methacrylate functionalized silanes, such as 3-methacryloyloxypropyl-trimethoxysilane.

The compositions according to the invention contain at least one radically polymerizable polyfunctional monomer. Polyfunctional monomers are understood to be compounds having two or more, preferably 2 to 4 and in particular 2 radically polymerizable groups. Monofunctional monomers accordingly have only one radically polymerizable group. Polyfunctional monomers have crosslinking properties and are therefore also referred to as crosslinking monomers. Preferred radically polymerizable groups are (meth)acrylate, (meth)acrylamide and vinyl groups.

According to the invention, a distinction is made between monomers containing acid groups and monomers which do not contain acid groups. The compositions according to the invention contain at least one monomer without acid groups and at least one monomer and/or oligomer with acid groups. According to the invention, compositions are preferred which contain monomers with and monomers without acid groups in a weight ratio of from 1:5 to 1:36, more preferably from 1:6 to 1:25 and most preferably from 1:7 to 1:20.

### Monomers Without Acid Group

Preferred are compositions comprising at least one polyfunctional (meth)acrylate, more preferably at least one polyfunctional and at least one monofunctional methacrylate, and most preferably at least one monofunctional and at least one difunctional methacrylate.

Preferred monofunctional (meth)acrylates are benzyl, tetrahydrofurfuryl or isobornyl (meth)acrylate, p-cumyl phenoxyethylene glycol methacrylate (CMP-1 E) and 2-([1,1′-biphenyl]-2-oxy)ethyl methacrylate (MA-836), tricyclodecane methyl (meth) acrylate, 2-(2-biphenyloxy)ethyl (meth)acrylate. CMP-1 E and MA-836 are particularly suitable.

According to one embodiment, the compositions according to the invention preferably comprise at least one functionalized monofunctional (meth)acrylate. Functionalized monomers are understood to be those monomers which, in addition to at least one radically polymerizable group, carry at least one functional group, preferably a hydroxyl group. Preferred functionalized mono(meth)acrylates are 2-hydroxyethyl-(meth)acrylate and hydroxyethylpropyl(meth)acrylate as well as 2-acetoxyethyl methacrylate. Hydroxyethyl methacrylate is particularly preferred. The monomers containing acid groups mentioned below are not functionalized monomers within the meaning of the invention.

Preferred di- and polyfunctional (meth)acrylates are bisphenol-A-dimethacrylate, Bis-GMA (an addition product of methacrylic acid and bisphenol-A-diglycidyl ether), ethoxy or propoxylated bisphenol-A-dimethacrylates, such as the bisphenol A dimethacrylate with 3 ethoxy groups (SR-348c, Sartomer) or 2,2-bis[4-(2-methacryloyloxypropoxy) phenyl]propane, urethanes of 2-(hydroxymethyl) acrylic acid methyl ester and diisocyanates, such as 2,2,4-trimethylhexamethylene diisocyanate or isophorone diisocyanate, UDMA (an addition product of 2-hydroxyethyl methacrylate and 2,2,4-trimethylhexamethylene-1,6-diisocyanate), tetramethylxylylene diurethane ethylene glycol di(meth)acrylate or tetramethylxylylene diurethane-2-methylethylene glycol di-(meth)acrylate (V380), di-, tri- or tetraethylene glycol dimethacrylate, trimethylolpropane trimethacrylate, pentaerythritol tetramethacrylate, and glycerol di- and trimethacrylate, 1,4-butane diol dimethacrylate, 1,10-decanediol dimethacrylate (D3MA), bis(methacryloyloxymethyl) tricyclo[5.2.1.0^{2,6}]decane (DCP), polyethylene glycol or polypropylene glycol dimethacrylates, such as polyethylene glycol 200 dimethacrylate or polyethylene glycol 400 dimethacrylate (PEG-200 or PEG-400 DMA) or 1,12-dodecanediol dimethacrylate. Bis-GMA, UDMA, V-380, triethylene glycol dimethacrylate (TEGDMA) and PEG-400-DMA (NK ester 9G) are particularly preferred.

The monomer tetramethylxylylene diurethane ethylene glycol di(meth)acrylate or tetramethylxylylene diurethane 2-methylethylene glycol diurethane di(meth)acrylate (V380) has the following formula:

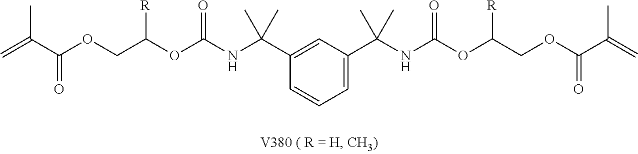

In the formula shown, the residues R are each independently H or CH₃, and the residues may have the same meaning or different meanings. Preferably, a mixture is used containing molecules in which both residues are H, molecules in which both residues are CH₃, and molecules in which one residue is H and the other residue is CH₃ , the ratio of H to CH₃ preferably being 7:3. Such a mixture is obtainable, for example, by reacting 1,3-bis(1-isocyanate-1-methylethyl)benzene with 2-hydroxypropyl methacrylate and 2-hydroxy ethyl methacrylate.

Other preferred difunctional monomers are radically polymerizable pyrrolidones, such as 1,6-bis(3-vinyl-2-pyrrolidonyl)hexane, or commercially available bisacrylamides such as methylene or ethylene bisacrylamide, as well as bis(meth)acrylamides, such as N,N′-diethyl-1,3-bis(acrylamido)propane, 1,3-bis(methacrylamido) propane, 1,4-bis(acrylamido)butane or 1,4-bis(acryloyl)piperazine, which can be synthesized by reacting the corresponding diamines with (meth)acrylic acid chloride. N,N′-Diethyl-1,3-bis(acrylamido) propane (V392) is particularly preferred. These monomers are characterized by high hydrolytic stability.

### Acid Group-Containing Monomers and Oligomers

The compositions according to the invention comprise at least one acidic, radically polymerizable monomer and/or at least one acidic oligomer, with compositions comprising at least one acidic, radically polymerizable monomer being preferred. Acidic monomers and oligomers are understood to mean monomers or oligomers containing at least one acid group, preferably a phosphoric ester, phosphonic acid or carboxyl group. Acidic monomers and oligomers are also referred to herein as adhesive components or adhesive monomers or adhesive oligomers. According to the present invention, those compositions are particularly preferred which contain at least one strongly acidic monomer. Strongly acidic monomers are monomers with a pKa value, at room temperature, of 0.5 to 4.0, more preferably 1.0 to 3.5 and most preferably 1.5 to 2.5.

Suitable monomers containing acid groups are COOH group-containing polymerizable monomers, preferably with a pKa value in the range of 2.0 to 4.0. 4-(Meth)-acryloyloxyethyltrimellitic anhydride, 10-methacryloyloxydecylmalonic acid, N-(2-hydroxy-3-methacryloyloxypropyl)-N-phenylglycine and 4-vinylbenzoic acid are preferred. Methacrylic acid (pKa=4.66) is excluded due to its low adhesion to the tooth structure.

Preferred monomers containing acid groups are phosphoric ester and phosphonic acid monomers, preferably with a pKa value in the range of 0.5 to 3.5. Particularly preferred are 2-methacryloyloxyethylphenyl hydrogen phosphate, 10-methacryloyloxydecyl dihydrogen phosphate (MDP), glycerol dimethacrylate dihydrogen phosphate or dipentaerythritol pentamethacryloyloxy phosphate, 4-vinylbenzylphosphonic acid, 2-[4-(dihydroxyphosphoryl)-2-oxa-butyl]-acrylic acid or hydrolysis-stable esters, such as 2-[4-(dihydroxyphosphoryl)-2-oxa-butyl]-acrylic acid 2,4,6-trimethylphenyl ester. MDP, 2-methacryloyloxyethylphenyl hydrogen phosphate and glycerol dimethacrylate dihydrogen phosphate are particularly preferred.

Oligomers are understood to be polymers with a degree of polymerization Pₙ of 2 to 100 (Pₙ=Mₙ/Mᵤ; Mₙ=number average polymer molar mass, Mᵤ=molar mass of the monomer unit). Acidic, radically polymerizable oligomers have at least one acid group, preferably a carboxyl group, and at least one radically polymerizable group, preferably at least one (meth)acrylate group and more preferably at least one methacrylate group.

Oligomers containing acid groups preferred according to the invention are oligomeric carboxylic acids, such as polyacrylic acid, preferably with a number average molecular weight Mₙ of less than 7200 g/mol, more preferably less than 7000 g/mol and most preferably less than 6800 g/mol, where Mₙ is preferably in a range from 800 to 7200 g/mol, more preferably from 500 to 7000 g/mol and most preferably from 500 to 6800 g/mol. Oligomeric carboxylic acids with (meth)acrylate groups are particularly preferred. These can be obtained, for example, by reacting oligomeric polyacrylic acid with glycidyl methacrylate or 2-isocyanatoethyl methacrylate.

Unless otherwise stated herein, the molar mass of oligomers and polymers is the number average molar mass Mₙ, determined by gel permeation chromatography (GPC).

Gel permeation chromatography (GPC) is a relative method in which molecules are separated based on their size, or more precisely, their hydrodynamic volume. The absolute molar mass is determined by calibration with known standards. Preferably, narrowly distributed polystyrene standards are used as calibration standards. These are commercially available. Styrene-divinylbenzene columns are used as separation material and tetrahydrofuran (THF) as eluent. Styrene-divinylbenzene columns are suitable for organic soluble synthetic polymers. The measurement is carried out with diluted solutions (0.05-0.2% by weight) of the polymers to be tested.

Alternatively, the number average molar mass can be determined by the well-known methods of freezing point depression (cryoscopy), boiling point elevation (ebullioscopy) or from the depression of the vapor pressure (vapor pressure osmometry). These are absolute methods that do not require calibration standards. Concentration series of 4 to 6 dilute polymer solutions with concentrations of 0.005 to 0.10 mol/kg are examined and then the measured values are extrapolated to a concentration of 0 mol/kg.

The compositions according to the invention also preferably contain water. It has been found that a water content of 1 to 7% by weight, particularly preferably 1 to 5% by weight, in each case based on the total mass of the composition, brings about an improvement in the bonding effect to dentin and enamel.

The compositions according to the invention further comprise at least one initiator for initiating the radical polymerization, preferably a photoinitiator. Preferred photoinitiators are benzophenone, benzoin and derivatives thereof, α-diketones or derivatives thereof, such as 9,10-phenanthrenequinone, 1-phenyl-propane-1,2-dione, diacetyl and 4,4′-dichlorobenzil. Particularly preferred are camphorquinone (CC) and 2,2-dimethoxy-2-phenyl-acetophenone and most preferably α-diketones in combination with amines as reducing agents, such as 4-(dimethylamino)-benzoic acid ethyl ester (EDMAB), N,N-dimethylaminoethyl methacrylate, N,N-dimethyl-sym.-xylidine or triethanolamine, are used. Further preferred are Norrish type I photoinitiators, especially acyl or bisacyl phosphine oxides and most preferably monoacyltrialkyl, diacyldialkylgermanium and tetraacylgermanium compounds, such as benzoyltrimethylgerman, dibenzoyldiethylgerman, bis(4-methoxybenzoyl)diethyl-german (Ivocerin®), tetrabenzoylgerman or tetrakis(o-methylbenzoyl)german. Mixtures of the various photoinitiators can also be used, such as bis(4-methoxy-benzoyl)diethylgerman or tetrakis(o-methylbenzoyl)german in combination with camphorquinone and 4-dimethylaminobenzoic acid ethyl ester.

Further preferred are compositions containing a redox initiator for initiating radical polymerization, preferably a redox initiator based on an oxidizing agent and a reducing agent. Preferred oxidizing agents are peroxides and in particular hydroperoxides. A particularly preferred peroxide is benzoyl peroxide. Preferred hydroperoxides are the low-odor cumene hydroperoxide derivatives disclosed in EP 3 692 976 A1, the oligomeric CHP derivatives disclosed in EP 21315089.9, and in particular 4-(2-hydroperoxypropan-2-yl)phenylpropionate and cumene hydroperoxide (CHP).

Preferred reducing agents for combination with peroxides are tertiary amines, such as N,N-dimethyl-p-toluidine, N,N-dihydroxyethyl-p-toluidine, p-dimethylaminobenzoic acid ethyl ester or other aromatic dialkylamines, ascorbic acid, sulfinic acids, thiols and/or hydrogen silanes.

Preferred reducing agents for combination with hydroperoxides are thiourea derivatives, in particular the compounds listed in EP 1 754 465 A1 in paragraph [0009]. Particularly preferred are methyl, ethyl, allyl, butyl, hexyl, octyl, benzyl, 1,1,3-trimethyl, 1,1-diallyl, 1,3-diallyl, 1-(2-pyridyl)-2-thiourea, acetyl, propanoyl, butanoyl, pentanoyl, hexanoyl, heptanoyl, octanoyl, nonanoyl, decanoyl, benzoylthiourea, and mixtures thereof. Very particularly preferred are acetyl, allyl, pyridyl and phenylthiourea as well as hexanoylthiourea and mixtures thereof as well as polymerizable thiourea derivatives, such as N-(2-methacryloyloxyethoxysuccinoyl)-thiourea and N-(4-vinylbenzoyl)-thiourea. In addition, a combination of one or more of said thiourea derivatives with one or more imidazoles may advantageously be used. Preferred imidazoles are 2-mercapto-1-methylimidazole or 2-mercaptobenzimidazole.

In addition to at least one hydroperoxide and at least one thiourea derivative, the compositions according to the invention may further comprise at least one transition metal compound for accelerating curing. Transition metal compounds suitable according to the invention are in particular compounds derived from transition metals having at least two stable oxidation states. Particularly preferred are compounds of the elements copper, iron, cobalt, nickel and manganese. These metals have the following stable oxidation states: Cu(I)/Cu(II), Fe(II)/Fe(III), Co(II)/Co(III), Ni(II)/Ni(III), Mn(II)/Mn(III). Compositions containing at least one copper compound are particularly preferred. The transition metal compounds are preferably used in catalytic amounts, more preferably in an amount of 10 to 200 ppm. These do not lead to discoloration of the dental materials. Because of their good monomer solubility, the transition metals are preferably used in the form of their acetylacetonates, 2-ethylhexanoates or THF adducts. Further preferred are their complexes with polydentate ligands such as 2-(2-aminoethylamino)ethanol, triethylenetetramine, dimethylglyoxime, 8-hydroxyquinoline, 2,2′-bipyridine or 1,10-phenanthroline. A particularly suitable initiator according to the invention is a mixture of cumene hydroperoxide (CHP) with at least one of the above-mentioned thiourea derivatives and copper(II) acetylacetonate. According to the invention, compositions which do not contain vanadium compounds are preferred.

The compositions according to the invention may also contain additional additives, in particular stabilizers, colorants, microbicidal agents, fluoride ion-releasing additives, such as fluoride salts, in particular NaF or ammonium fluoride, or fluorosilanes, optical brighteners, plasticizers and/or UV absorbers.

According to the invention, preferred compositions are those containing the following ingredients:
a) 25 to 80% by weight, preferably 30 to 75% by weight and more preferably 40 to 70% by weight of at least one coated FAS and/or radiopaque glass filler,
b) 0 to 50% by weight, preferably 0 to 40% by weight and more preferably 2 to 30% by weight of one or more further fillers, preferably metal oxides,
c) 1 to 15% by weight, preferably 2 to 12% by weight and more preferably 3 to 10% by weight of at least one monomer containing acid groups,
d) 5 to 40% by weight, preferably 8 to 30% by weight and more preferably 10 to 25% by weight of at least one polyfunctional monomer without acid groups,
e) 0 to 10% by weight, preferably 0 to 8% by weight and more preferably 1 to 5% by weight of one or more oligomeric carboxylic acids,
f) 0 to 20% by weight, preferably 0 to 15% by weight and more preferably 1 to 10% by weight of one or more monofunctional monomers without acid groups,
g) 0.1 to 8% by weight, preferably 0.5 to 6% by weight and more preferably 1 to 5% by weight of at least one initiator for the radical polymerization,
h) 0 to 20% by weight, preferably 0.01 to 10% by weight and more preferably 0.1 to 7% by weight of water and
i) 0.01 to 5% by weight, preferably 0.1 to 3% by weight and more preferably 0.1 to 2% by weight of additives, in each case based on the total mass of the composition.

The initiator can be a redox initiator, a photoinitiator or an initiator for dual curing. The amounts mentioned include all initiator components, i.e. the initiators themselves and, if applicable, reducing agents, transition metal compound, etc. According to the invention, compositions containing at least one redox initiator or at least one redox initiator and at least one photoinitiator are preferred.

Compositions containing a redox initiator are also referred to as self-curing. They are preferably used in the form of two spatially separated components, i.e. as a 2-component system (2C system). Oxidizing and reducing agents are incorporated into separate components of the composition. One component, the so-called catalyst paste, contains the oxidizing agent, preferably a peroxide or hydroperoxide, and optionally a photoinitiator, and the second component, the so-called base paste, contains the corresponding reducing agent, optionally a photoinitiator and optionally catalytic amounts of a transition metal compound. Polymerization is initiated by mixing the components. Compositions containing both a redox initiator and a photoinitiator are referred to as dual-curing.

According to the invention, 2-component systems are preferred. They are preferably self-curing or dual-curing. The pastes are mixed together shortly before use, preferably with a double-push syringe.

The catalyst paste preferably has the following composition:
a) 25 to 80% by weight, preferably 30 to 75% by weight and more preferably 40 to 20 70% by weight of at least one coated FAS and/or glass filler,
b) 0 to 50% by weight, preferably 0 to 40% by weight and more preferably 2 to 30% by weight of one or more further fillers, preferably metal oxides,
c) 2 to 30% by weight, preferably 4 to 24% by weight and more preferably 6 to 20% by weight of at least one monomer containing acid groups,
d) 5 to 40% by weight, preferably 8 to 30% by weight and more preferably 10 to 25% by weight of at least one polyfunctional monomer without acid groups,
e) 0 to 20% by weight, preferably 0 to 16% by weight and more preferably 1 to 10% by weight of one or more oligomeric carboxylic acids,
f) 0 to 20% by weight, preferably 0 to 15% by weight and more preferably 1 to 10% by weight of one or more monofunctional monomers without acid groups,
g) 0.01 to 16% by weight, preferably 0.02 to 12% by weight and more preferably 0.03 to 10% by weight of at least one peroxide and/or hydroperoxide and optionally at least one photoinitiator,
h) 0 to 20% by weight, preferably 0.01 to 10% by weight and more preferably 0.1 to 7% by weight of water, and
i) 0.001 to 5% by weight, preferably 0.002 to 3% by weight and more preferably 0.0051 to 2% by weight of one or more additives, in each case based on the total mass of the catalyst paste.

The base paste preferably has the following composition:
a) 25 to 80% by weight, preferably 30 to 75% by weight and more preferably 40 to 70% by weight of at least one FAS and/or glass filler,
b) 0 to 50% by weight, preferably 0 to 40% by weight and more preferably 2 to 30% by weight of one or more further fillers, preferably metal oxides,
c) not applicable
d) 5 to 40% by weight, preferably 8 to 30% by weight and more preferably 10 to 25% by weight of at least one polyfunctional monomer without acid groups,
e) not applicable
f) 0 to 20% by weight, preferably 0 to 15% by weight and more preferably 1 to 10% by weight of one or more monofunctional monomers without acid groups,
g) 0.01 to 16% by weight, preferably 0.02 to 12% by weight and more preferably 0.1 to 10% by weight of at least one suitable reducing agent and optionally at least one photoinitiator,
h) 0 to 20% by weight, preferably 0.01 to 10% by weight and more preferably 0.1 to 7% by weight of water, and
i) 0.001 to 5% by weight, preferably 0.002 to 3% by weight and more preferably 0.005 to 2% by weight of one or more additives, in each case based on the total mass of the base paste.

The coating of the fillers prevents interactions of the fillers with acidic monomers or oligomers. According to the invention, acid monomers and oligomers are only present in the catalyst paste, so that uncoated FAS fillers and/or X-ray opaque glass fillers are preferably used to prepare the base paste, wherein the above-described FAS fillers and/or X-ray opaque glass fillers in uncoated form are preferred.

For application, the catalyst and base paste are preferably mixed together in approximately equal proportions. They are therefore particularly suitable for application with a double-push syringe.

Double-push syringes have two separate cylindrical chambers for holding the base paste and catalyst paste. The components are pushed out of the chambers simultaneously by two interconnected pistons and are preferably forced through a mixing nozzle and mixed together therein. For pressing out the pastes, the syringe can be inserted into a so-called hand dispenser, which facilitates handling of the syringes.

The compositions according to the invention are characterized by high storage stability and improved transparency, preferably greater than 10%, and good self-adhesion to enamel/dentin. They are particularly suitable as dental materials for intraoral use by the dentist for the restoration of damaged teeth (therapeutic use), especially as dental cements, coating or veneering materials, filling composites and most particularly as luting cements. The transparency is determined in the manner described in the examples.

The coating makes it possible to produce stable dental composites with conventional radiopaque fillers, which allow the production of composites with a high transparency but are disadvantageous due to their acid sensitivity. It was expected that when the fillers were coated, residual surface silanol groups (Si—OH) formed from the silanes of formula I would lead to networks of hydrogen bonds and thus cause a significant increase in viscosity. Surprisingly, it was found that the coating according to the invention does not lead to an increase in the thickening effect of the fillers. In addition, the coated fillers were found to improve the miscibility of catalyst and base paste, thus considerably facilitating the preparation of homogeneous materials.

For the treatment of damaged teeth, these are preferably prepared in a first step by the dentist. Subsequently, at least one composition according to the invention is applied to or into the prepared tooth. Thereafter, the composition can be cured directly, preferably by irradiation with light of a suitable wavelength, for example when restoring cavities. Alternatively, a dental restoration, for example an inlay, onlay, veneer, crown, bridge, framework or dental ceramic, is placed in or applied to the prepared tooth. Subsequent curing of the composition is preferably achieved by light and/or self-curing. Thereby, the prosthesis is bonded to the tooth.

The compositions according to the invention can also be used as extraoral materials (non-therapeutic), for example in the fabrication or repair of dental restorations. They are also suitable as materials for the fabrication and repair of inlays, onlays, crowns or bridges.

For the production of dental restorations such as inlays, onlays, crowns or bridges, at least one composition according to the invention is formed into the desired dental restoration in a manner known per se and then cured. The curing can be done by light, self-curing or preferably thermally.

In the repair of dental restorations, the compositions according to the invention are applied to the restoration to be repaired, for example to repair gaps or to join fragments, and then cured.

The invention is explained in more detail below with reference to figures and examples.

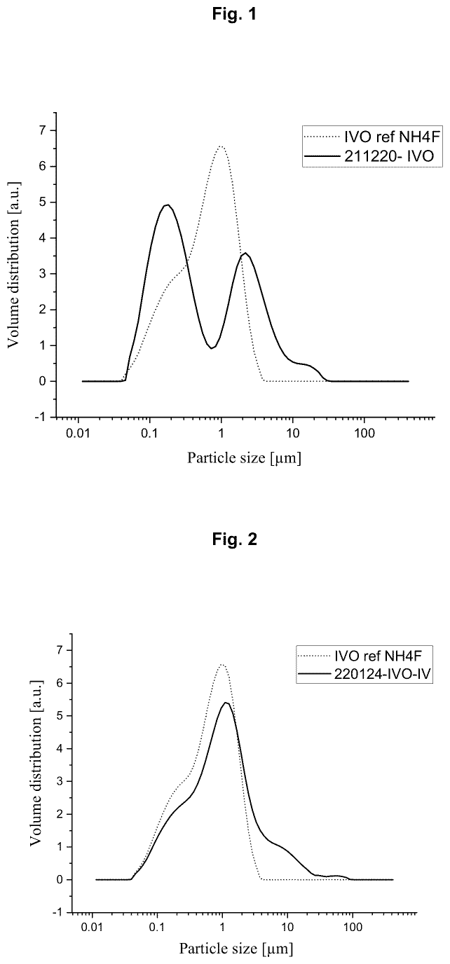

FIGS. 1 to 3 each show the particle size distribution of fillers coated according to the invention in comparison with the uncoated starting materials.

FIG. 4 shows the decrease in the concentration of the acidic monomer MDP as a function of the storage time in a composite paste with the coated glass filler from Example 1 ( [symbol] ) compared to a composite paste with the uncoated glass filler ( [symbol] ). In the paste with the uncoated filler, the MDP concentration decreases rapidly, while the paste according to the invention is stable over the test period.

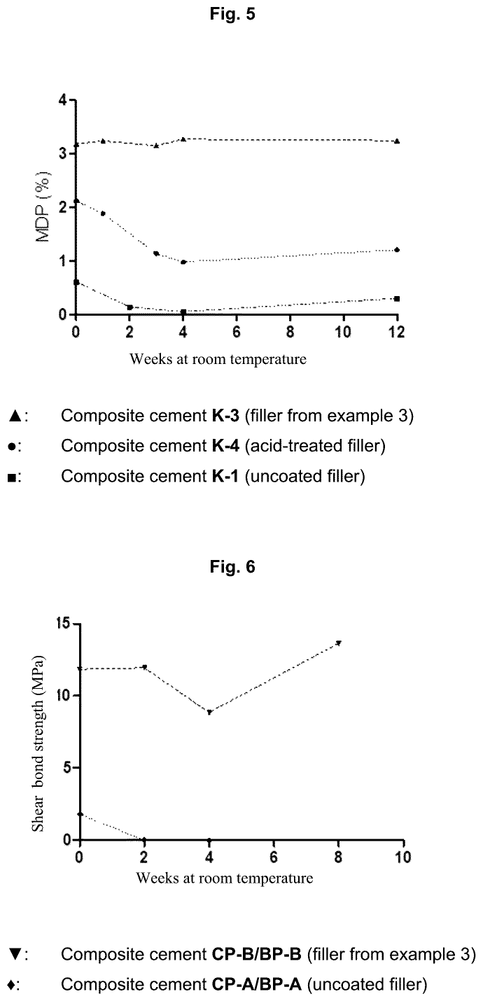

FIG. 5 shows the decrease in the concentration of the acidic monomer MDP as a function of storage time in a composite paste with the coated glass filler from Example 3 ( [symbol] ) compared to a composite paste with the uncoated glass filler ( [symbol] ) and a composite paste with an acid-treated filler ( [symbol] ). The paste with the acid-treated filler exhibits significantly higher stability compared to the paste with the uncoated filler. The stability of the paste according to the invention with the coated filler is again considerably improved. No decrease in MDP concentration was observed over the test period.

FIG. 6 shows the dentin shear bond strength of a two-component composite cement with the coated filler from example 1 (upper curve) compared to a cement with uncoated filler (lower curve) as a function of their storage time. The cement according to the invention achieves high adhesion values even after several weeks of storage, while the comparative cement no longer produces an adhesive effect after only two weeks.

### EXAMPLES

### Example 1

### Preparation of a Coated Glass Filler (Solids Content 10% by Weight)

A mixture of 120.0 g of the radiopaque glass filler GM 27884, (Schott AG; mean particle size 1 μm; specific surface area (BET DIN ISO 9277) 3.9 m²/g; composition (wt %): Al₂O₃: 10, B₂O₃: 10, BaO: 25, and SiO₂: 55), 1080.0 g of the solvent isopropoxyethanol (IPE), 750.0 g of tetraethoxysilane (TEOS), and 376.0 g of deionized water were stirred in a 5 L double-walled reactor with a KPG stirrer and reflux condenser at a stirring speed of 500 revolutions per minute (rpm) at 23° C. The solids content was 10 wt %. To prepare a catalyst solution, 0.325 g of ammonium fluoride was dissolved in 250 mL of deionized water and stirred overnight. 12.83 g of this NH₄F solution was added to the above reaction mixture. The mixture was then heated to 100° C. and stirred further at 100° C. for 3 hours. Then the mixture was distributed into four 500 ml centrifuge containers and centrifuged at 2000 rpm for 15 minutes. The separated filler particles were washed at least twice with isopropanol and centrifuged again. Subsequently, the washed filler particles were dried in a vacuum drying cabinet at 60° C. for 12 hours. The obtained sample was designated as 211220-IVO.

To determine the particle size distribution, 0.1 g of the coated particles (sample 211220-IVO) were added to 50 g of deionized water and stirred for two hours on a magnetic stirrer. The sample was then treated in an ultrasonic bath for 30 minutes. In parallel, 0.1 g uncoated filler particles (sample IVO ref NH₄F) were dispersed in 50 g water in the manner described. The filler IVO ref NH₄F was prepared in the manner described above, but no silane (TEOS) was added. The particle size distribution was determined by dynamic light scattering (DLS) (Malvern Mastersizer 2000 with dispersing unit Hydro 2000S). The particle size distribution (particle sizes distribution histogram) is shown in FIG. 1. For the coated sample 211220-IVO a bimodal particle size distribution with peak maxima at 0.2 μm and 2 μm was determined.

The refractive index of the filler used (n_{D}=1.526) was not changed by the coating. The refractive indices of the coated and the uncoated filler were determined by the immersion method (Carl-Zeiss Abbé refractometer model A 72901; immersion oils: 2-hydroxy-3-phenoxypropyl acrylate (n_{D}=1.526) and cedar oil (n_{D}=1.504)).

### Example 2

Determination of the Ion Release of the Fillers from Example 1 in Acid

For the determination of the barrier effect, 2 g of the coated filler particles from Ex. 1 (sample 211220-IVO) were dispersed in 50 g of 1 M HCl and stirred for 1 h. The particles were then removed by centrifugation. After centrifugation of the leached particles, the Al, B, Ba content of the centrifugate was determined. For this purpose, the samples were diluted with ultrapure water (Millli-Q®, Merck Company) in a ratio of 1:10 or 1:100, depending on the Al, B, Ba ion concentration in the solution. The analysis was performed by Inductively Coupled Plasma Optical Emission Spectrometry (ICP-OES; instrument: Horiba Jobin Yvon Ultima2 with Conikal atomizer; measurement parameters: Pressure: 2.71 bar, Flow: 0.73 l/min, Emission wavelengths: Al: λ=396.152 nm, B: λ=208.957 nm, Ba: λ=455.403 nm). The following concentrations were determined: Al: 3.97 mg/L; B: 2.74 mg/L; and Ba: 23.0 mg/L.

In contrast, an analogous leaching test with the non-coated filler IVO ref NH₄F gave the following concentrations: Al: 226.9 mg/L; B: 126.7 mg/L and Ba: 1060 mg/L.

A comparison of the values shows that the coating of the fillers reduced the leaching of Al ions by 98.2% by weight and of Ba ions by 97.8% by weight.

### Example 3

Production of a Coated Glass Filler with Reduced Silane Concentration

A mixture of 120.0 g of the radiopaque glass filler GM 27884, 1080.0 g of IPE, 250.0 g of TEOS, and 125.3 g of deionized water was stirred in a 5 L double-walled reactor at a stirring rate of 500 rpm at 23° C. The solids content was 10 wt %. 4.28 g of the NH₄F solution from Example 1 was added to the reaction mixture, the mixture was then heated to 100° C. and stirred at 100° C. for 24 hours. The mixture was then centrifuged as described in Example 1, and the separated filler particles were washed and dried (Sample 220124-IVO). The particle size distribution was determined analogously to Example 1 and is shown in FIG. 2. The DLS measurement showed a monomodal particle size distribution, which largely corresponded to that of the uncoated reference sample IVO ref NH₄F. The peak maximum was at approx. 4 μm.

The refractive index of the filler used (no=1.526) was not changed by the coating.

The leaching test analogous to Example 2 resulted in the following concentrations: Al: 1.75 mg/L; B: 1.43 mg/L; and Ba: 18.87 mg/L. The leaching of Al ions was reduced by 99.2% by weight and that of Ba ions by 98.2% by weight by the coating, both compared to the reference sample IVO ref NH₄F.

### Example 4

### Preparation of a Coated Glass Filler (Solids Content 20% by Weight)

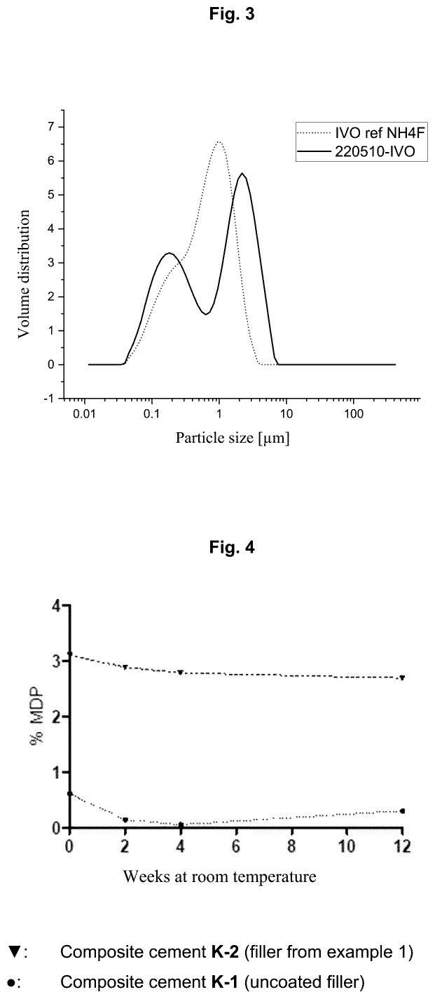

A mixture of 150.0 g of the radiopaque glass filler GM 27884, 600.0 g of IPE, 312.50 g of TEOS, and 156.66 g of deionized water was stirred in a 5 L double-walled reactor at a stirring rate of 500 rpm at 23° C. 5.35 g of the NH₄F solution from Example 1 was added to reaction mixture, the mixture was then heated to 100° C. and stirred at 100° C. for 24 hours. The mixture was then centrifuged as described in Example 1, and the separated filler particles were washed and dried (Sample 220510-IVO). The particle size distribution was determined analogously to Example 1 and is shown in FIG. 3. The DLS measurement showed a bimodal particle size distribution with peak maxima at 0.2 μm and 2.2 μm, similar to sample 211220-IVO in example 1.

The leaching test analogous to Example 2 resulted in the following concentrations: Al: 5.067 mg/L; B: 1.91 mg/L; and Ba: 10.49 mg/L. The leaching of Al ions was reduced by 97.8% by weight and that of Ba ions by 99.0% by weight by the coating, both compared to the reference sample IVO ref NH₄F.

### Example 5

### Production of Acid-Treated Fillers (General Specification According to EP 21218194.5)

In two centrifuge plastic vessels, each with a filling volume of 1 L, 150 g of the filler to be treated and 350 g of 1.0 molar hydrochloric acid are poured in and stirred for 1 h at room temperature on a magnetic stirring plate. After removing the magnetic stirrer, the mixtures are centrifuged in a centrifuge (Hettich Silenta RS) for 5 min at 3000 rpm, during which the filler settles. The liquid is separated and a sample of the liquid is taken for X-ray fluorescence (XRF) analysis. The pH of the liquid is 1-2. The separated filler is then dispersed in 400 ml of deionized water and the dispersion is centrifuged again for 5 min at 3000 rpm. Then the wash solution is separated by decantation. The washing procedure is repeated until the pH increases to 5 or more in the last wash (approximately three times). After washing, the acid-treated filler is dried in the vacuum drying cabinet at 50° C. until constant weight is obtained, and then silanized. For silanization, 12 g of 3-methacryloyloxypropyltrimethoxysilane (Silane A-174, Sigma Aldrich) is added to the filler (185 g) and then mixed for 15 min (Turbola mixer, Willy A. Bachofen AG). Then 5 g deionized water is added and mixed again for 15 min. The filler is then sieved through a 90 μm plastic sieve, allowed to rest for 24 h and then dried in a drying oven at 50° C. for 3 days until no free silane is detectable (gas chromatography).

### Example 6

### Determination of the Storage Stability of Glass Fillers Coated According to the Invention

Composite cement pastes K-1 to K-4 were prepared with the compositions given in Table 1:

TABLE 1
Composition of composite pastes [wt. %].

| Component | K-1*) | K-2 | K-3 | K-4*) |
|---|---|---|---|---|
| uncoated glass filler¹⁾ | 65 | — | — | — |
| coated glass filler from Ex. 1 | — | 65 | — | — |
| coated glass filler from Ex. 3 | — | — | 65 | — |
| acid treated glass filler²⁾ | — | — | — | 65 |
| HDK 2000³⁾ | 4 | 4 | 4 | 4 |
| MDP⁴⁾ | 3.29 | 3.29 | 3.29 | 3.29 |
| TEGDMA⁵⁾ | 8.53 | 8.53 | 8.53 | 8.53 |
| NK ester 9G⁶⁾ | 1.90 | 1.90 | 1.90 | 1.90 |
| V-392⁷⁾ | 11.68 | 11.68 | 11.68 | 11.68 |
| Water | 5.58 | 5.58 | 5.58 | 5.58 |
| BHT⁸⁾ | 0.03 | 0.03 | 0.03 | 0.03 |

*)Comparative example
¹⁾Non-treated glass filler GM 27884, 1.0 μm, unsilanized (Schott AG)
²⁾Glass filler GM 27884, 1.0 μm, treated with acid according to Ex. 5
³⁾Fumed silica (specific surface area (BET) 120 m²/g, Wacker AG)
⁴⁾10-Methacryloyloxydecyl dihydrogen phosphate (Orgentis)
⁵⁾Triethylene glycol dimethacrylate
⁶⁾Polyethylene glycol 400 dimethacrylate (Kowa Europa GmbH)
⁷⁾N,N′-Diethyl-1,3-bis(acryl amido)-propane (Ivoclar Vivadent AG)
⁸⁾2,6-Di-tert-butyl-p-cresol

The pastes were stored for a period of several weeks at room temperature and the content of the acidic monomer MDP was repeatedly determined by HPLC. An HPLC Ultimate 3000 instrument (ThermoFisher Scientific) with a 125×4 Nucleodur 100-5 C18ec column and with UV/VIS detector (220 nm) was used for HPLC measurement. The samples to be measured were dissolved in methanol. Elution was performed using the eluents water with 0.01 mol/L o-phosphoric acid and acetonitrile according to a standard gradient program (the acetonitrile percentage was continuously increased to 100%). The results are shown in FIGS. 4 and 5 and show the change in the content of MDP in the composite pastes over time.

The results shown in FIG. 4 demonstrate a markedly improved storage stability of the composite cement K-2 compared to the composite cement K-1 with the uncoated glass filler GM 27884. K-1 shows a marked decrease of the available MDP already after 2 to 4 weeks. The differences at time 0 are due to the fact that the acidic monomer reacts very rapidly with the untreated filler during paste manufacture.

The results shown in FIG. 5 demonstrate a significantly improved storage stability of the composite cement K-4, which contains an acid-treated glass filler according to European patent application No. EP 21218194.5, compared to the composite cement K-1 with uncoated filler. The composite cement K-3 according to the invention shows a further improved stability. Even after storage for 12 weeks, no change in the MDP concentration can be detected.

### Example 7

### Measurement of the Dentin Shear Bond Strength of Composite Cements According to the Invention

Dual-curing two-component composite cements were prepared. Each cement comprised a catalyst paste and a base paste. The compositions of the pastes are given in Tables 2 and 3. Dentin bond strength as a function of storage time was determined. For the measurement of dentin adhesion, bovine teeth were embedded in a plastic cylinder with an addition-curing vinyl polysiloxane (Dreve Company) in such a way that the dentin and the plastic were in one plane. The tooth surfaces ground with sandpaper (grit size 400) were rinsed with lukewarm water and pre-tempered to 37° C. The dentin surfaces were then rinsed with water. The dentin surfaces were blotted dry, the catalyst pastes were each mixed with the corresponding base paste in a 1:1 ratio and then applied to the tooth surface. At the same time, the underside of a plug made of a cured dental composite material (Tetric Evo-Ceram, Ivoclar Vivadent AG) was wetted with the cement and placed as centrally as possible on the dentin surface. The tooth was then clamped with the composite plug facing upwards in an Ultradent clamping device so that the fixation mandrel was centered on the Tetric Evo-Ceram plug. The excess luting cement was then carefully removed immediately and the Ultradent fixture with the clamped tooth was stored in the drying cabinet at 37° C. for 15 minutes. The plugs were then relieved, kept in water at 37° C. for 24 hours, and then stored in the drying cabinet at 37° C. for 24 hours. To measure shear bond strength, the plugs were sheared at 23° C. using a Zwick testing machine according to the Ultradent method (EN ISO 35 29022, year 2013), and the shear bond strength was obtained as the quotient of the breaking force and the bond area. The results are shown in FIG. 6.

TABLE 2
Composition of catalyst pastes CP-A and CP-B

| Component | CP-A* | CP-B |
|---|---|---|
| MDP⁴⁾ | 3.41 | 3.41 |
| TEGDMA⁵⁾ | 10.72 | 10.72 |
| NK ester 9G⁶⁾ | 2.38 | 2.38 |
| UDMA¹⁴⁾ | 14.68 | 14.68 |
| DBPO¹⁷⁾ | 0.97 | 0.97 |
| BHT⁸⁾ | 0.031 | 0.031 |
| Water | 0.831 | 0.827 |
| uncoated glass filler ¹⁾ | 63.43 | — |
| Filler from Ex. 3 | — | 63.43 |
| HDK 2000³⁾ | 2.10 | 2.11 |
| HDK H-15¹⁶⁾ | 1.44 | 1.44 |

*Comparison example

TABLE 3
Composition of the base pastes BP-A and BP-B

| Component | BP-A | BP-B |
|---|---|---|
| TEGDMA⁵⁾ | 8.62 | 8.54 |
| NK ester 9G⁶⁾ | 2.53 | 2.51 |
| UDMA¹⁴⁾ | 12.60 | 12.50 |
| DABA¹⁵⁾ | 0.600 | 1.502 |
| Camphorquinone | 0.076 | 0.075 |
| 4-Dimethylaminobezoic acid ethyl ester | 0.152 | 0.151 |
| Phase transfer catalyst⁹⁾ | 0.203 | 0.201 |
| Color pigment¹⁰⁾ | 0.00074 | 0.00076 |
| TEMPO¹¹⁾ | 0.00077 | 0.00080 |
| Water | 0.6501 | 0.644 |
| Glass filler G018-056, 7 μm, 5% sil.¹²⁾ | 69.98 | 69.64 |
| Glass filler G018-056, 1 μm, 5% sil.¹³⁾ | 3.08 | 3.05 |
| HDK 2000³⁾ | 1.49 | 1.48 |

¹⁾ Non-treated glass filler GM 27884, 1.0 μm, unsilanized (Schott AG)
³⁾Fumed silica (specific surface area (BET) 120 m²/g, Wacker AG)
⁴⁾ 10-Methacryloyloxydecyl dihydrogen phosphate (Orgentis)
⁵⁾Triethylene glycol dimethacrylate
⁶⁾Polyethylene glycol 400 dimethacrylate (Kowa Europa GmbH)
⁸⁾ 2,6-Di-tert-butyl-p-cresol
⁹⁾Benzyltributylammonium chloride
¹⁰⁾2,5-Dihydroxyterephthalic acid diethyl ester (Lumilux LZ Flu Blue, Riedel-de Haën AG)
¹¹⁾2,2,6,6-Tetramethylpiperidinyloxyl (CAS number 2564-83-2)
¹²⁾Glass filler G018-056, 7 μm, 5% sil.: 24 wt % Al₂O₃, 23 wt % SiO₂, 16.5 wt % CaO, 16 wt % CaF₂, 11.5 wt % BaO, 8 wt % P₂O₅, 2 wt % Na₂O, 5% silane; weight average particle diameter 7 μm (Schott AG, Mainz)
¹³⁾Glass filler G018-056, 1 μm, 5% sil.: 24 wt % Al₂O₃, 23 wt % SiO₂, 16.5 wt % CaO, 16 wt % CaF₂, 11.5 wt % BaO, 8 wt % P₂O₅, 2 wt % Na₂O, 5% silane; weight average particle diameter 1 μm (Schott AG, Mainz)
¹⁴⁾1,6-Bis-[2-methacryloyloxyethoxycarbonyl amino]-2,2,4-trimethylhexane
¹⁵⁾N,N-diethyl-3,5-di-tert-butylaniline
¹⁶⁾ Fumed silica (specific surface area (BET) 130-170 m²/g, Wacker AG)
¹⁷⁾ 50% Dibenzoyl peroxide (DBPO) dispersed in 50% dicyclohexyl phthalate (DBPO: BP 50 FT; United Initiator)

The results shown in FIG. 6 demonstrate that the cement CP-B/BP-B according to the invention, which was obtained by mixing the catalyst paste CP-B and the base paste BP-B, achieved high bond strengths even after storage for several weeks. The catalyst paste CP-B according to the invention contains the strongly acidic monomer MDP and an X-ray opaque Ba glass filler coated according to the invention. The adhesion values measured demonstrate that the coating of the filler effectively suppresses undesirable reactions with the acidic monomer. The base pastes do not contain acid monomers, so that uncoated glass fillers were used here. The comparative cement CP-A/BP-A with uncoated filler showed no more adhesion already after storage for only 2 weeks.

1. A radically polymerizable composition comprising
at least one polyfunctional radically polymerizable monomer without an acid group,
at least one acid group-containing radically polymerizable monomer and/or acid oligomer,
at least one fluoroaluminosilicate glass filler and/or radiopaque glass filler and
at least one initiator for the radical polymerization,
characterized in that the fluoroaluminosilicate glass filler and/or radiopaque glass filler is coated with a silicic acid (hetero)polycondensate.

2. The composition of claim 1, wherein the fluoroaluminosilicate glass filler and/or radiopaque glass filler comprises a coating formed by hydrolytic condensation of one or more silanes of the general formula I
SiXₐR₄₋ₐ Formula I
in which the variables have the following meanings:
R=C₁-C₂₀-alkyl or C₂-C₁₀-alkenyl, where alkyl and alkenyl residues may comprise one or more O or S atoms, ester, amide, amino or urethane groups, C₆-C₁₄-aryl or C₆-C₁₅-arylalkyl,
X=halogen, hydroxy, C₁-C₁₀-alkoxy, C₁-C₆-acyloxy,
a=2, 3 or 4,
and optionally one or more further hydrolytically condensable compounds of boron, aluminum, titanium and/or phosphorus and/or precondensates derived from the abovementioned compounds.

3. The composition according to claim 2, wherein the amount of silanes of formula I is from 10 to 100% by weight based on the total amount of hydrolytically condensable compounds.

4. The composition according to claim 1, wherein the coated fluoroaluminosilicate glass filler and/or radiopaque glass filler is obtainable by dispersing the filler to be coated in one or more silanes of formula I and optionally further hydrolytically condensable compounds, or a solution of the silane(s) of formula I and optionally further hydrolytically condensable compounds in a suitable solvent, subsequently adding water, stirring the resulting mixture and subsequently separating and drying the filler.

5. The composition according to claim 1, which comprises
a radiopaque glass filler having the composition (wt %): SiO₂: 20-80; B₂O₃: 5-15; BaO or SrO: 0-30; Al₂O₃: 5-20; CaO and/or MgO: 0-20; Na₂O, K₂O, Cs₂O: 0-10 each; WO₃: 0-20; La₂O₃: 0-10; ZrO₂: 0-15; P₂O₅: 0-10; Ta₂O₅, Nb₂O₅ or Yb₂O₃: 0-5 and CaF₂ or SrF₂: 0-10, or SiO₂: 50-75; B₂O₃: 5-15; BaO or SrO: 2-30; Al₂O₃: 5-15; CaO and/or MgO: 0-10 and Na₂O: 0-10,
and/or
a fluoroaluminosilicate glass filler having the composition (wt %): SiO₂: 20-35; Al₂O₃: 15-35; BaO or SrO: 0-25; CaO: 0-20; ZnO: 0-15; P₂O₅: 5-20; fluoride 3-18, or SiO₂: 20-30; Al₂O₃: 20-30; BaO or SrO: 5-15; CaO: 5-18; P₂O₅: 5-15; Na₂O: 2-10; and CaF₂: 5-20,
where all data refer to the total mass of the glass and where all components except fluorine are calculated as oxides.

6. The composition according to claim 1, wherein the polyfunctional radical polymerizable monomer is selected from bisphenol-A-dimethacrylate, Bis-GMA (an addition product of methacrylic acid and bisphenol-A-diglycidyl ether), ethoxy or propoxylated bisphenol-A-dimethacrylates, such as bisphenol-A-dimethacrylate with 3 ethoxy groups or 2,2-bis[4-(2-methacryloyloxypropoxy)-phenyl] propane, UDMA (an addition product of 2-hydroxyethylmethacrylate- and 2,2,4-trimethylhexamethylene-1,6-diisocyanate), tetramethylxylylene diurethane ethylene glycol di(meth)acrylate or tetramethylxylylene diurethane-2-methylethylene glycol diurethane di(meth)acrylate (V380), di-, tri- or tetraethylene glycol dimethacrylate, trimethylolpropane trimethacrylate, penta-erythritol tetramethacrylate, and glycerol di- and -trimethacrylate, 1,4-butanediol dimethacrylate, 1,10-decanediol dimethacrylate (D3MA), bis(methacryloyloxymethyptricyclo[5.2.1.0^{2,6}]decane (DCP), polyethylene glycol or polypropylene glycol dimethacrylates, such as polyethylene glycol 200-dimethacrylate (PEG-200-DMA) or polyethylene glycol 400-dimethacrylate (PEG-400-DMA), 1,12-dodecanediol dimethacrylate, urethanes of 2-(hydroxymethyl) acrylic acid and diisocyanates, such as 2,2,4-trimethylhexamethylene diisocyanate or isophorone diisocyanate, pyrrolidones, such as 1,6-bis(3-vinyl-2-pyrrolidonyl)-hexane, bisacrylamides, such as methylene or ethylene bisacrylamide, bis(meth) acrylamides, such as N,N′-diethyl-1,3-bis(acrylamido)propane, 1,3-bis(methacrylamido) propane, 1,4-bis(acrylamido)butane or 1,4-bis(acryloyl)piperazine, and mixtures thereof.

7. The composition according to claim 1, comprising as acid group-containing monomer at least one monomer selected from monomers containing a phosphoric ester group or phosphonic acid group, preferably 2-methacryloyloxyethylphenyl hydrogen phosphate, 10-methacryloyloxydecyl dihydrogen phosphate (MDP), glycerol dimethacrylate dihydrogen phosphate, dipentaerythritol pentamethacryloyloxyphosphate, 4-vinylbenzylphosphonic acid, 2-[4-(dihydroxyphosphoryl)-2-oxa-butyl]acrylic acid and/or 2-[4-(dihydroxyphosphoryl)-2-oxa-butyl]acrylic acid-2,4,6-trimethyl phenyl ester, and/or 4-(meth)acryloyloxyethyltrimellitic anhydride, 10-methacryl-oyloxydecylmalonic acid, N-(2-hydroxy-3-methacryloyloxypropyl)-N-phenyl glycine and/or 4-vinylbenzoic acid.

8. The composition according to claim 4, comprising as acidic oligomer a polyacrylic acid having a number average molecular weight of less than 7200 g/mol.

9. The composition according to claim 1, which additionally comprises at least one monofunctional radically polymerizable monomer without an acid group selected from benzyl, tetrahydrofurfuryl (meth)acrylate, isobornyl (meth)acrylate, p-cumyl-phenoxyethyleneglycol methacrylate (CMP-1E) and 2-([1,1′-biphenyl]-2-oxy)ethyl methacrylate (MA-836), tricyclodecane methyl (meth)acrylate, 2-(2-biphenyloxy)ethyl (meth)acrylate, 2-hydroxyethyl-(meth)acrylate, hydroxyethylpropyl (meth)acrylate), 2-acetoxy ethyl methacrylate, and mixtures thereof.

10. The composition according to claim 1, comprising
(a) 25 to 80% by weight of at least one coated fluoroaluminosilicate glass filler and/or radiopaque glass filler,
b) 0 to 50% by weight of one or more further fillers, preferably metal oxides,
c) 1 to 15% by weight of at least one monomer containing acid groups,
d) 5 to 40% by weight of at least one polyfunctional monomer without acid groups,
e) 0 to 10% by weight of one or more oligomeric carboxylic acids,
f) 0 to 20% by weight of one or more monofunctional monomers without acid groups,
g) 0.1 to 8% by weight of at least one initiator for the radical polymerization,
h) 0 to 20% by weight of water, and
i) 0.01 to 5% by weight of additives,
in each case based on the total mass of the composition.

11. The composition according to claim 1, comprising a catalyst paste and a base paste, wherein the catalyst paste comprises
a) 25 to 80% by weight of at least one coated fluoroaluminosilicate glass filler and/or glass filler,
b) 0 to 50% by weight of one or more further fillers,
c) 2 to 30% by weight of at least one monomer containing acid groups,
d) 5 to 40% by weight of at least one polyfunctional monomer without acid groups,
e) 0 to 20% by weight of one or more oligomeric carboxylic acids,
f) 0 to 20% by weight of one or more monofunctional monomers without acid groups,
g) 0.01 to 16% by weight of at least one peroxide and/or hydroperoxide and optionally at least one photoinitiator,
h) 0 to 20% by weight of water, and
i) 0.001 to 5% by weight of one or more additives, in each case based on the total mass of the catalyst paste,
and wherein the base paste comprises
a) 25 to 80% by weight of at least one FAS and/or glass filler,
b) 0 to 50% by weight of one or more further fillers,
c) not applicable
d) 5 to 40% by weight of at least one polyfunctional monomer without acid groups,
e) not applicable
f) 0 to 20% by weight of one or more monofunctional monomers without acid groups,
g) 0.01 to 16% by weight of at least one suitable reducing agent and optionally at least one photoinitiator,
h) 0 to 20% by weight of water, and
i) 0.001 to 5% by weight of one or more additives, in each case based on the total mass of the base paste.

12. The composition according to claim 1 for therapeutic use as a dental material comprising a dental cement, coating or veneering material, filling composite or luting cement.

13. The composition according to claim 1 for non-therapeutic use comprising preparing or repairing a dental restoration selected from an inlay, onlay, crown or bridge.

14. A process for the preparation of a coated fluoroaluminosilicate glass filler or glass filler, which process comprises
(i) dispersing the filler to be coated in one or more silanes of formula I and optionally further hydrolytically condensable compounds or, preferably, in a solution of the silane or silanes of formula I and optionally further hydrolytically condensable compounds in a suitable solvent,
(ii) subsequently adding water,
(iii) stirring the resulting mixture,
(iv) subsequently separating and drying the filler.

15. The process according to claim 14, further comprising mixing the coated fluoroaluminosilicate glass filler or glass filler with one or more monomers to produce a radically polymerizable composition.
